(19) United States
(12) Patent Application Publication
(10) Pub. No.: US 2013/0064345 A1
(43) Pub. Date: Mar. 14, 2013

(54) RADIOGRAPHY IMAGE CAPTURING AND PATIENT LIMB POSITIONING ASSEMBLY AND METHOD OF OBTAINING A RADIOGRAPHIC IMAGE OF A PATIENT

(71) Applicant: Serge Kaska, Encinitas, CA (US)

(72) Inventor: Serge Kaska, Encinitas, CA (US)

(21) Appl. No.: 13/396,279

(22) Filed: Feb. 14, 2012

Related U.S. Application Data
(60) Provisional application No. 61/442,390, filed on Feb. 14, 2011.

(51) Int. Cl. A61B 6/04; A61B 6/00

(52) U.S. Cl. 378/62; 378/208

(57) ABSTRACT

A radiography image capturing and patient limb positioning assembly and method of obtaining a radiographic image of a patient is provided. The assembly includes a radiolucent patient limb positioning member configured to substantially contour to a portion of a patient's body. Further, the assembly includes a housing configured to receive a radiography image capturing media, wherein the housing is operably fixed to the radiolucent member. The method includes positioning the assembly in substantially contoured relation with a portion of the patient's body. Then, releasing the assembly so that it is automatically maintained in position through engagement with the patient's body. Further, directing x-rays through the region of the patient to be imaged, through the radiolucent member and onto the radiographic image capturing media.

### CROSS-REFERENCE TO RELATED APPLICATION

This application claims the benefit of U.S. Provisional Application Ser. No. 61/442,390, filed Feb. 14, 2011, which is incorporated herein by reference in its entirety.

### BACKGROUND OF THE INVENTION

1. Technical Field

This invention relates generally to radiographic examination apparatus, and more particularly to positioning apparatus for radiography imaging media and patient limbs.

2. Related Art

Obtaining radiographic images, e.g. an x-ray image, of a patient can prove challenging for a technician, while at the same time requiring the technician to remain exposed to potentially harmful x-rays. In addition to these drawbacks the technician faces, the process of obtaining radiographic images can prove to be painful for the patient, particularly if the image is of an area requiring a patient's injured limb to be manipulated to order to properly position a radiography cassette.

One such case that commonly presents the aforementioned drawbacks, to the technician, as well as the patient, is when a patient's injured shoulder region requires imaging. A complete x-ray series of the shoulder, required for proper diagnosis and treatment of traumatic shoulder injuries, consists of a 3 view x-ray series including an AP (Anterior Posterior) view (external rotation); a lateral view (internal rotation), and a Axillary view, Abduction. The patient with an injured shoulder frequently presents to the emergency department with the arm held to the chest (adducted and internally rotated). This is the position for the lateral view image and can be obtained without manipulation. The AP and axillary views require manipulation and commonly two x-ray technicians; one to hold the limb and x-ray cassette while the other shoots the radiograph; otherwise one technician must hold the limb x-ray cassette and shoot the x-ray image simultaneously. This procedure is time consuming and technically challenging for the technician, and requires multiple manipulations of a patient's injured limb, leading to undue discomfort during the diagnostic process. These obstacles frequently result in inadequate images being obtained and an incomplete x-ray series. Any one skilled in the art of orthopedic trauma surgery is aware of the difficulty in obtaining an adequate 3 view x-ray series of the injured shoulder in a busy emergency department. Therefore, a need exists for a x-ray positioning device which aids in obtaining consistent, accurate x-ray projections for the shoulder and potentially other limbs; permits secure immobilization of the limb in the appropriate position; holds the x-ray cassette to enable the x-ray technician to be free from harmful x-ray exposure, and permits the patient maximum comfort and security to the injured limb with minimal manipulation.

### SUMMARY OF THE INVENTION

In accordance with one aspect of the invention, a radiography image capturing and patient limb positioning assembly is provided. The assembly includes a radiolucent patient limb positioning member configured to substantially contour to a portion of a patient's body. Further, the assembly includes a housing configured to receive a radiography image capturing media, wherein the housing is fixed to the radiolucent member.

In accordance with another aspect of the invention, the radiolucent patient limb positioning member is provided as a compliant material.

In accordance with another aspect of the invention, the radiolucent patient limb positioning member is a closed or open cell foam.

In accordance with another aspect of the invention, the housing is configured to receive an x-ray photographic film cassette.

In accordance with another aspect of the invention, the housing is configured to receive a digital x-ray sensor.

In accordance with another aspect of the invention, the radiolucent patient limb positioning member has opposite sides with one of the sides being contoured to substantially conform with a patient's forearm and the other of the sides being contoured to substantially conform with the patient's torso.

In accordance with another aspect of the invention, the radiolucent patient limb positioning member has a plurality of retention straps configured to immobilize a patient's limb to the radiolucent patient limb positioning member.

According to another aspect of the invention, a method of obtaining a radiographic image of a patient is provided. The method includes providing an assembly including a housing with a radiography image capturing media disposed therein with the housing being operably fixed to a radiolucent member configured to substantially contour to a portion of the patient. Further, positioning the assembly in substantially contoured relation with a portion of the patient's body and then releasing the assembly so that it is automatically maintained in position through engagement with the patient's body. Then, directing x-rays through the region of the patient to be imaged, through the radiolucent member and onto the radiography image capturing media.

Accordingly, the invention overcomes the limitations discussed above by providing a positioning and imaging assembly that allows the desired images of a patient to be obtained without needless repetition; without causing the patient unnecessary pain, and while allowing the imaging technician to be clear of the potentially harmful x-rays.

### BRIEF DESCRIPTION OF THE DRAWINGS

These and other aspects, features and advantages of the present invention will become more readily appreciated when considered in connection with the following detailed description of presently preferred embodiments and best mode, appended claims and accompanying drawings, in which:

FIG. 1 is a partial side view of a patient using a radiography imaging media housing and patient limb positioning member assembly constructed in accordance with one presently preferred aspect of the invention;

FIG. 2 is a front view of the patient and radiography imaging media housing and patient limb positioning member assembly of FIG. 1;

FIG. 3 is a perspective view of the radiography imaging media housing and patient limb positioning member assembly of FIG. 1;

FIG. 4 is a top view of the radiography imaging media housing and patient limb positioning member assembly of FIG. 1;

FIG. 5 is a view similar to FIG. 3 of a patient using a radiography imaging media housing and patient limb positioning member assembly constructed in accordance with another presently preferred aspect of the invention; and

FIG. 6 is a top view of the radiography imaging media housing and patient limb positioning member assembly of FIG. 5.

### DETAILED DESCRIPTION OF PRESENTLY PREFERRED EMBODIMENTS

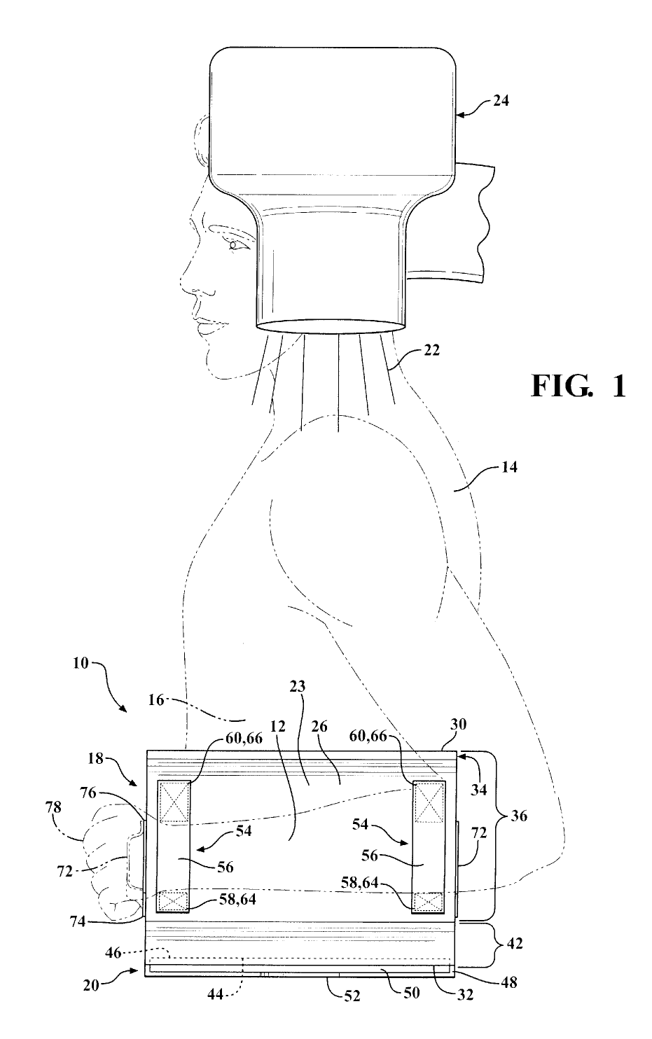

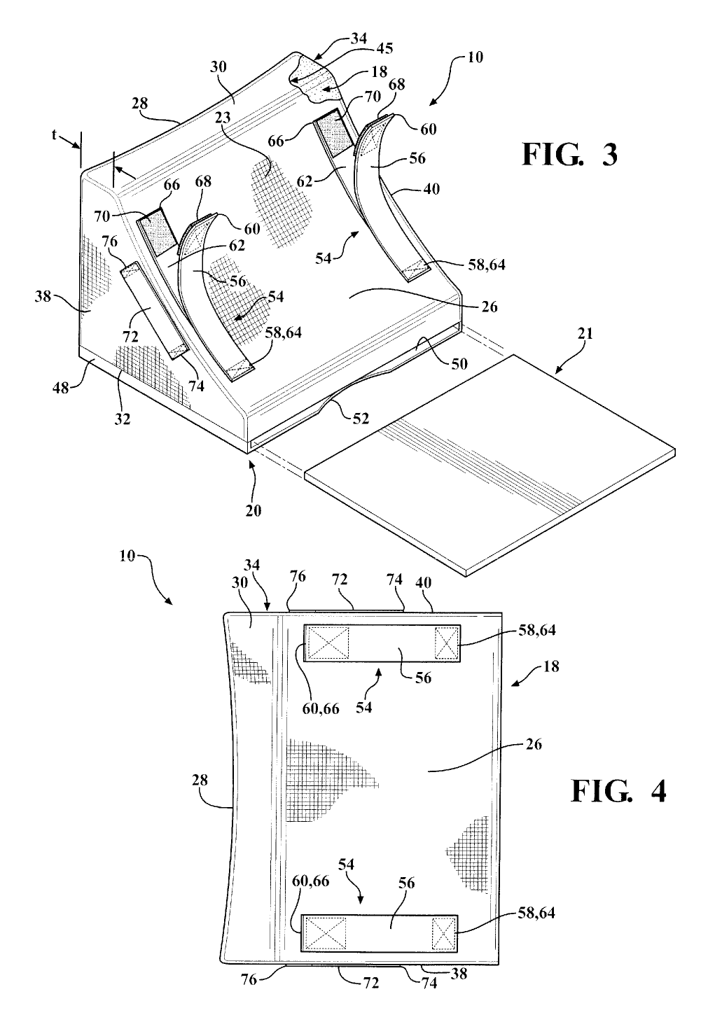

Referring in more detail to the drawings, FIGS. 1-4 illustrate a radiography image capturing and patient limb positioning assembly, referred to hereafter simply as assembly 10, constructed in accordance with one aspect of the invention. The assembly 10 is shown disposed in an axillary position between a forearm 12 of a patient 14 and the patient's thorax 16. The assembly 10 includes a radiolucent patient limb positioning member, referred to hereafter simply as positioning member 18, configured to contour or substantially contour to a portion of a patient's body, shown as conforming to the side 16 and forearm 12 of the patient 14. Further, the assembly 10 includes a housing 20 configured to receive a radiography image capturing media 21 (FIG. 3), wherein the housing 20 is fixed to the positioning member 18. As such, the assembly 10 functions as an all inclusive positioning and imaging apparatus that allows a technician to both reliably maintain the position the patient's arm in a desired orientation with minimal discomfort to the patient 14, while at the same time being automatically maintained in position through engagement with the patient's body. Accordingly, the radiography image capturing media 21 is automatically positioned in a desired location to reliably obtain a clear image of the patient's injured shoulder region without requiring the technician to be subjected to the potentially harmful x-rays 22 emitted from a X-ray emitting device 24. This assembly 10 allows for obtaining both the antero posterior view the imaging media posterior to the shoulder, and the axillary image with the imaging media 21 inferior to the shoulder at the base of the positioning member 18. Further, given the symmetry of the assembly 10, it can be used readily on either side of the patient 14, and thus, is reversible.

The positioning member 18 is constructed of a radiolucent material that allows the assembly 10 to conform or substantially conform to the patient's body. For example, the positioning member 18 can be fabricated from a compliant foam material, such as a closed or open cell foam, or it can be constructed as an inflatable member, for example. When used in an axillary position, the positioning member 18 is preferably configured having opposite sides 26, 28, with one of the sides 26 having a concave contour to conform or substantially conform with the patient's forearm 12 and the other of the sides 28 having a concave contour to conform or substantially conform with the patient's torso 16. In addition to being configured having concave surfaces as shown, the radiolucent material is preferably compliant to facilitate providing a comfortable fit between the assembly 10 and the patient 14.

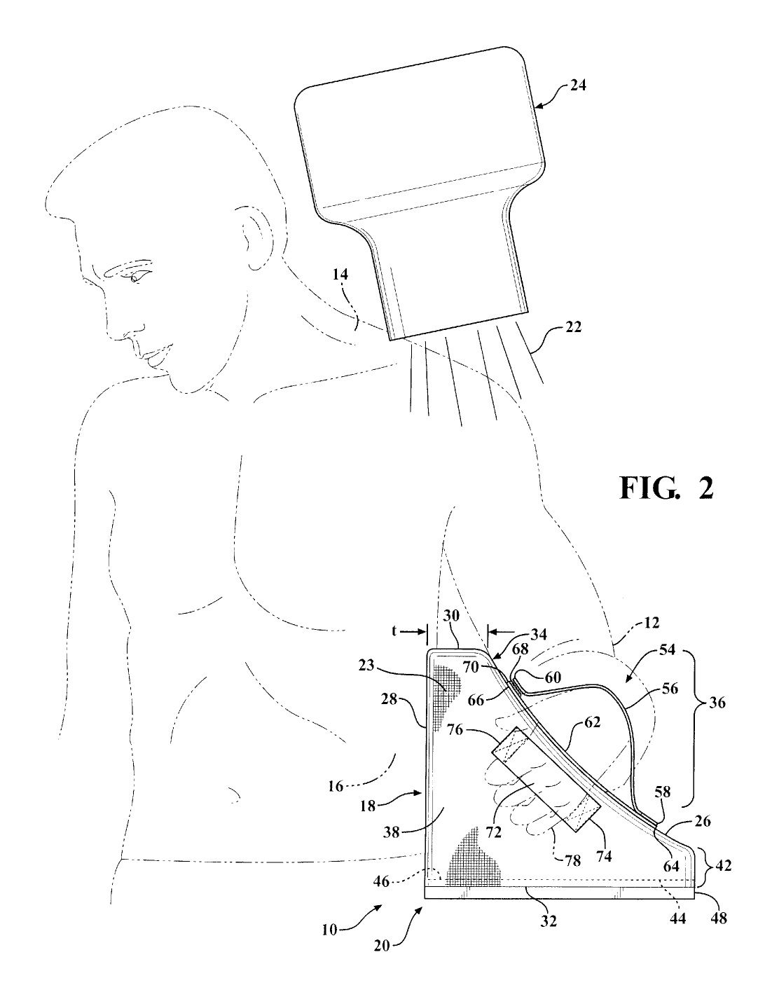

The positioning member 18 has an upper surface 30 and a base, also referred to as lower surface 32, with the opposite sides 26, 28 extending therebetween. To further promote comfort to the patient and properly locating the positioning member 18 in use, the upper surface 30 can be formed having a peak 34 with a reduced thickness (t) extending between the opposite sides 26, 28. As such, an upper portion 36 of the positioning member 18 has a generally convex or generally frustroconical shape, as view from one of the ends 38, 40 of the member 18 (FIGS. 2 and 3). As such, the upper portion 36 has a gradually upwardly tapered configuration wherein the opposite sides 26, 28 converge toward the peak 34. Accordingly, the upper portion 36 has a reduced tapering thickness that facilitates fitting the positioning member 18 closely within the axillary region of the patient, such as shown in FIG. 2, while a lower portion 42 can remain generally block-shaped, taking on the configuration of a rectangular prism.

The lower surface 32 of the generally block shaped lower portion 42 is configured for operably fixed attachment to the housing 20. To facilitate positioning and fixing the housing 20 to the positioning member 18, the lower surface 32 of the positioning member 18 can have a recessed cavity or pocket 44 formed therein (FIGS. 1 and 2), if desired. The recessed cavity or pocket 44, if provided, is shaped to receive at least a portion of the housing 20 in a close fit therein. To fix the housing 20 to the positioning member 18, any suitable adhesive, mechanical fastener or bond/weld joint can be used. Otherwise, it should be recognized that the housing 20 could be operably fixed to a flat or substantially flat surface of the lower surface 32.

The positioning member 18 can be contained in an outer layer 23 of health care grade material, such as a durable elastic material or fabric, e.g. Nylon containing Spandex®, for example, or any other suitable material health care compliant material, such as a urethane coating or film, whether elastic or inelastic. The outer layer 23 can be formed from one or more pieces of material stitched to one another, such that an inner cavity 45 is formed for close receipt of the positioning member 18. Upon disposing the compliant positioning member 18 in the cavity 45 of the outer layer 23, the outer layer 23 can be closed to fully encase the positioning member 18 in the fully enclosed cavity 45, such as via stitching, or any suitable closure mechanism could be used, e.g. sealable flap including fasteners, e.g. hook and loop, or otherwise. Accordingly, the positioning member 18 is contained in the outer layer 23 much like a pillow in an enclosed pillow case, however, it should be recognized that the outer layer 23 is free or substantially free from wrinkles.

The housing 20 can be constructed from any suitable material, though for single use disposable reasons, it is preferably constructed from a lightweight polymeric material. The housing 20, if partially received in the recessed pocket 44, has an upper portion 46 configured for close receipt in the recessed pocket, while a lower portion 48 extends outwardly from the lower surface 32 of the positioning member 18. The lower portion 48 has a cavity 50 for receipt of the radiography image capturing media 21, whether a X-ray photographic cassette or a digital, filmless radiography sensor. Accordingly, the radiography image capturing media 21 can be readily disposed into the housing 20 and removed therefrom with the housing remaining fixed to the positioning member 18. To facilitate insertion and removal of the media 21 from the housing 20, the lower surface of the housing can have a scalloped or recessed region 52 located generally midway between the opposite ends 38, 40, thereby allowing the media 21 to be readily grasped during insertion and removal. Of course, if the positioning member 18 is fully encased in the outer layer 23, then the housing 20 is fixed directly to the fabric outer layer 23.

To facilitate maintaining the assembly 10 in fixed relation to the patient 14, the radiolucent positioning member 18 has at least one, and shown as a plurality of retention straps 54 configured to releasably secure the assembly 10 to the patient 14. The retention straps 54 are operably fixed to the concave side 26 of the radiolucent positioning member 18, shown as being stitched or otherwise attached to the outer layer 23, with each retention strap including an elongate first portion 56 configured to extend over the patient's limb, shown as the forearm 12. The first portion 56 has one end 58 fixed to the outer layer 23, and thus, operably to the radiolucent positioning member 18 and another free end 60 configured for releasable operable attachment to the radiolucent positioning member 18. In the embodiment shown, the retention straps 54 also include an elongate second portion 62 fixed along its full length to the outer layer 23, and thus, operably fixed to the concave side 26. One end 64 of the second portion 62 is fixed with the end 58 of the first portion 56 to the outer layer 23. The second portion 62 extends in fixed relation to the outer layer 23 from the end 64 upwardly toward the peak 34 to an opposite free end 66. Each of the ends 60, 66 of the respective first and second portions 56, 62 have fasteners configured for releasable attachment to one another, shown as hook and loop type fasteners 68, 70, respectively. As such, in use, the forearm 12 is rested over the fixed second portion 62 and the first portion 56 is wrapped over the forearm 12 to bring the fasteners 68, 70 into releasably fixed relation with one another.

To further facilitate comfort and positioning of the assembly 10 against the patient 14, the ends 38, 40 of the radiolucent positioning member 18 each have a grasping strap 72. The grasping straps 72 are fixed at their opposite ends 74, 76 to the outer layer 23, such as via stitching or adhesive, and thus, operably to the ends 38, 40 of the positioning member 18. The straps 72 have free central portions located for ready access and grasping by a hand 78 of the patient 14. Of course, in use, only one of the grasping straps 72 is grasped by the patient 14, depending on which side of the patient 14 the assembly 10 placed. Accordingly, the assembly 10 is assured of remaining properly positioned during imaging and maintains the position the patient's arm in its desired orientation with minimal discomfort to the patient 14.

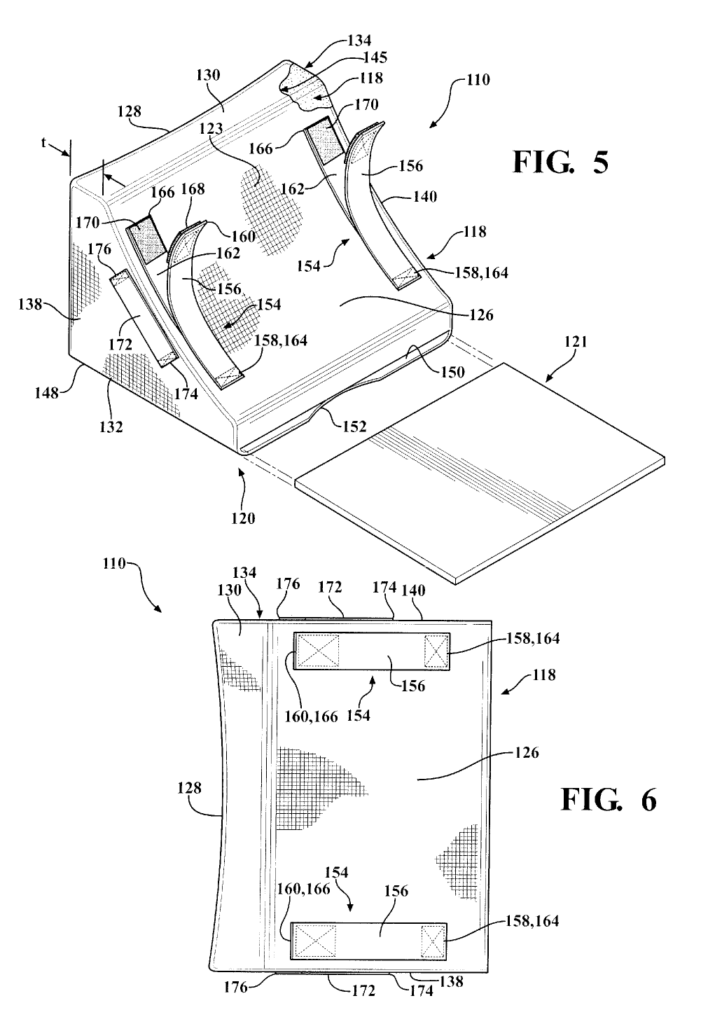

FIGS. 5-6 illustrate a radiography image capturing and patient limb positioning assembly, referred to hereafter simply as assembly 110, constructed in accordance with another aspect of the invention, wherein the same reference numerals as used above, offset by a factor of 100, are used to identify like features. Accordingly, although possibly not discussed in detail hereafter or otherwise mentioned, it is to be understood that the reference numerals offset by 100 and illustrated in FIG. 5-6 are as discussed above, unless otherwise stated below.

The assembly 110 is configured substantially the same as discussed above with regard to the assembly 10, however, the assembly 110 the outer layer 123 of health care grade fabric, such as a durable elastic material, e.g. Nylon containing Spandex®, for example, or any other suitable material health care compliant material, elastic or inelastic, not only encases an inner radiolucent positioning member 118, configured as described above with regard to the radiolucent positioning member 18, but also provides a housing 120 configured for receipt of the image capturing media 121. Accordingly, the housing 120 is not provided from a rigid, separate piece of material, as discussed above with regard to the housing 20, but rather, is provided as an integral, monolithic flexible component of the outer layer 123. As such, the housing 120 is formed as a pocket 150 in the outer layer 123, wherein the pocket 150 is separate and external to the cavity 145 in which the radiolucent positioning member 118 is received. Accordingly, the outer layer 123 provides both an enclosed cavity 145 for receipt of the radiolucent positioning member 118 as well as an open pocket or cavity 150 configured for receipt of the image capturing media 121.

In accordance with another aspect of the invention, a method of obtaining a radiographic image of a patient is provided. The method includes providing an assembly 10, 110 including a housing 20, 120 with a radiographic image capturing media 21, 121 disposed therein with the housing 20, 120 being operably fixed to a radiolucent member 18, 118 configured to substantially contour to a portion of the patient. Then, positioning the assembly 10, 110 in substantially contoured relation with a portion of the patient's body and releasing the assembly 10, 110 so that it is automatically maintained in position through engagement with the patient's body without aid of a technician. Further yet, directing x-rays through the region of the patient to be imaged, through the radiolucent member 18, 118 and onto the radiographic image capturing media 21, 121.

The method can further include releasably fixing a forearm of the patient to a first contoured side 26, 126 of the radiolucent member 18, 118 and bringing a second contoured side 28, 128 of the radiolucent member 18, 118 into substantially contoured abutment with the patient's torso. Further yet, the method can include fixing the forearm of the patient to the first contoured side 26, 126 with at least one retention strap 54, 154 that is operably fixed to the radiolucent member 18, 118 and bringing opposite ends 58, 60; 158, 160 of the at least one retention strap 54, 154 into releasable attachment with one another.

In accordance with another aspect of the invention, the method can further include providing an outer layer 23, 123 of material having an enclosed cavity 45, 145 bounding the radiolucent member 18, 118. Further, the method can include forming the housing 120 as an open cavity or pocket 150 from a monolithic piece of the outer layer 123 for releasable receipt of the radiographic image capturing media 121.

Many modifications and variations of the present invention are possible in light of the above teachings. It is, therefore, to be understood that the invention may be practiced otherwise than as specifically described, and that the scope of the invention is defined by any ultimately allowed claims.

What is claimed is:

1. A radiography image capturing and patient limb positioning assembly, comprising:
a radiolucent patient limb positioning member configured to substantially contour to a portion of a patient's body; and
a housing configured to receive a radiographic image capturing media, said housing being operably fixed to said radiolucent member.

2. The assembly of claim 1 wherein said radiolucent patient limb positioning member is a compliant material.

3. The assembly of claim 2 wherein said radiolucent patient limb positioning member is foam.

4. The assembly of claim 1 wherein said housing is configured to received an x-ray photographic film cassette.

5. The assembly of claim 1 wherein said housing is configured to received a digital x-ray sensor.

6. The assembly of claim 1 wherein said radiolucent patient limb positioning member has opposite sides, a first one of said sides being contoured to substantially conform with a patient's forearm and a second one of said sides being contoured to substantially conform with the patient's torso.

7. The assembly of claim 6 wherein said first side has at least one retention strap operably fixed thereto and configured to releasably fix the forearm to the radiolucent patient limb positioning member.

8. The assembly of claim 7 wherein said at least one retention strap has opposite ends configured for releasable attachment to one another.

9. The assembly of claim 1 wherein said radiolucent patient limb positioning member has opposite ends, at least one of said ends having a grasping strap operably fixed thereto and located for ready access and grasping by a hand of a patient.

10. The assembly of claim 1 further including an outer layer of material encasing said radiolucent patient limb positioning member.

11. The assembly of claim 10 wherein said outer layer forms said housing.

12. The assembly of claim 10 wherein said outer layer has an enclosed cavity and an open pocket, said enclosed cavity being configure to encase said radiolucent patient limb positioning member and said open pocket being configured for receipt of the radiographic image capturing media.

13. A method of obtaining a radiographic image of a patient, comprising:
providing an assembly including a housing with a radiographic image capturing media disposed therein with the housing being operably fixed to a radiolucent member configured to substantially contour to a portion of the patient;
positioning the assembly in substantially contoured relation with a portion of the patient's body;
releasing the assembly so that it is automatically maintained in position through engagement with the patient's body without aid of a technician; and
directing x-rays through the region of the patient to be imaged, through the radiolucent member and onto the radiographic image capturing media.

14. The method of claim 13 further including releasably fixing a forearm of the patient to a first contoured side of the radiolucent member and bringing a second contoured side of the radiolucent member into substantially contoured abutment with the patient's torso.

15. The method of claim 14 further including fixing the forearm to the first contoured side with at least one retention strap that is operably fixed to the radiolucent member.

16. The method of claim 15 further including bringing opposite ends of the at least one retention strap into releasable attachment with one another.

17. The method of claim 13 further including providing an outer layer of material having an enclosed cavity bounding the radiolucent member and forming the housing as an open pocket configured from the outer layer material for releasable receipt of the radiographic image capturing media.
